(19) United States
(12) Patent Application Publication
(10) Pub. No.: US 2022/0249264 A1
(43) Pub. Date: Aug. 11, 2022

(54) Inflatable Dilation Device

(71) Applicant: Tevar (PTY) LTD, Observatory (ZA)

(72) Inventors: Braden Sydney Clive VAN BREDA, Cape Town (ZA); Preyen Agasthian PERUMALL, Cape Town (ZA); Reno Kochaeppen CHACKO, Cape Town (ZA); Fadi Nkoma FAKIH, Cape Town (ZA); Roman GOTTARDI, Salzburg (AT); Peter ZILLA, Cape Town (ZA); Deon BEZUIDENHOUT, Cape Town (ZA)

(73) Assignee: Tevar (PTY) LTD, Observatory (ZA)

(21) Appl. No.: 17/626,734

(22) PCT Filed: Aug. 27, 2020
(86) PCT No.: PCT/ZA2020/050044

§ 371 Date: Jan. 12, 2022

(30) Foreign Application Priority Data
Sep. 13, 2019 (GB) 1913216.6

(51) Int. Cl. A61F 2/958; A61F 2/24; A61F 2/89; A61F 2/915; A61M 25/10; A61M 29/00

(52) U.S. Cl. CPC A61F 2/958; A61F 2/2418; A61F 2/243; A61F 2/89; A61F 2/915; A61M 25/1011; A61M 29/00; A61F 2002/91558; A61F 2002/91566; A61M 2025/1086; A61M 2025/1093; A61M 25/1002

(57) ABSTRACT

An inflatable dilatation device includes: (i) a tubular frame including a first frame member that zigzags circumferentially; and (ii) an elongate inflatable first balloon. The elongate inflatable first balloon: (i) is secured to the first frame member; and (ii) zigzags along the first frame member, such that inflation of the first balloon causes the first balloon and the first frame member circumferentially to expand in unison from a contracted condition to an expanded condition.

### BACKGROUND

The present invention relates to an inflatable dilatation device. More specifically, the present invention relates to an inflatable dilatation device comprising a tubular frame and an inflatable balloon. Even more specifically, the present invention relates to an inflatable dilatation device that comprises: a frame that includes a member that zigzags circumferentially; and an inflatable balloon secured to the zigzagging frame member.

Various expandable frames and stent arrangements are known. For instance:
WO2019/086958 “Expandable sealing skirt technology for leak-proof endovascular prosthesess”, WO2016/013006 “Pulmonary artery implant apparatus and methods of use thereof”, WO2003/057078 “Intravascular stent and method of use”, US2018/0110634 “Endoluminal device and method”, US2013/0166010 “Hybrid balloon-expandable/self-expanding prosthesis for deployment in a body vessel and method of making”, US2006/0259137 “Minimally invasive valve replacement system”, US2004/0127972 “Indwelling stent and living organ dilator”, US2001/0044648 “Radially-expandable stent and delivery system”, U.S. Pat. No. 6,013,854 “Indwelling stent and the method for manufacturing the same”, EP0808613 “Tubular prosthesis made of curable material”, EP1799152 “Stent” and EP1729684 “Stent deployment device” describe frames or stents comprising members that zigzag to facilitate radial expansion of the frame or stent;
WO2018/158635 “Stented valve”, WO2017/165840 “Vascular flow diversion”, WO2014/008460 “Methods, devices, and systems for postconditioning with clot removal”, WO2012/047308 “Alternating circumferential bridge stent design and methods for use thereof”, US2005/0131512 “Stent delivery catheter, US2016/0113789 “Stent with flexible hinge” and CA2462479 “Modified delivery device for coated medical devices” describe frames or stents that comprise: a first member that zigzags to facilitate radial expansion of the frame or stent; and second members that extend longitudinally from apexes defined by the first member; and
WO2017/151569 “Perfusion balloon with internal valve”, WO2016/013006 “Pulmonary artery implant apparatus and methods of use thereof”, WO2014/158816 “Catheter system with balloon-operated filter sheath and fluid flow maintenance”, US2015/0272732 “Reinforced inflatable medical devices”, US2012/0143239 “Devices and methods for removing clots”, US2012/0109179 “Intravasculature devices and balloons for use therewith”, U.S. Pat. No. 5,985,307 “Device and method for non-occlusive localized drug delivery” and U.S. Pat. No. 5,458,575 “Perfusion catheter having a cylindrical array of balloons” describe either: inflatable balloons that are longitudinally aligned; or an inflatable balloon that zigzags circumferentially to form a tube.

A drawback of known inflatable dilatation devices that include a frame an an inflatable balloon is that, upon expansion of the dilatation device, relative movement the balloon and adjacent portions of the frame requires either: the balloon to stabilise itself through contact with adjacent portions of the balloon; or links that extend between portions of the balloon. Known dilatation devices that include an inflatable balloon that zigzags circumferentially about a tubular frame are insufficiently stable if the balloon is secured to, and supported by the frame alone.

It is an object of the present invention to address this drawback by providing an inflatable dilatation device that includes:
a tubular frame comprising a first member that zigzags circumferentially; and
a zig-zagging inflatable balloon that follows, and is secured to the first frame member,
   thereby to ensure that expansion of the dilatation device consequent to inflation of the balloon results in reduced relative movement of the balloon on the one hand and portions of the frame that support the balloon on the other hand.

### SUMMARY OF THE INVENTION

According to a preferred embodiment of the invention, there is provided an inflatable dilatation device that includes:
a tubular frame including a first frame member that zigzags circumferentially; and
an elongate inflatable first balloon that:
   is secured to the first frame member; and
   zigzags along the first frame member,
      such that inflation of the first balloon causes the first balloon and the first frame member circumferentially to expand in unison from a contracted condition to an expanded condition.

Typically, the frame further includes a set of second frame members that extend longitudinally in a first axial direction from apexes on a first side of the first frame member.

Generally, the frame further includes a third frame member that: zigzags circumferentially; and is axially spaced from the second side of the first frame member.

Preferably, the frame further includes a set of fourth frame members that extend longitudinally between: longitudinally aligned apexes on a first side of the first and third frame members; and longitudinally aligned apexes on a second side of the first and third frame members.

Typically, the inflatable dilatation device further includes an elongate inflatable second balloon that:
is secured to the third frame member; and
zigzags along the third frame member,
   such that inflation of the third balloon causes the third balloon and the third frame member circumferentially to expand in unison from a contracted condition to an expanded condition.

Generally, the frame further includes a set of fifth frame members that extend longitudinally in a second axial direction from apexes on a second side of the third frame member.

Preferably:
the apexes on both sides of the first balloon correspond to the apexes on both sides of the first frame member; and
the apexes on both sides of the second balloon correspond to the apexes on both sides of the third frame member.

Typically, the tubular frame further includes:
a first ring at a first axial end of the tubular frame; and
a second ring at a second axial end of the tubular frame,
   which first and second rings do not expand in sympathy with expansion of the first or second frame members.

Generally:
the set of second frame members extend from the first frame member to the first ring; and
the set of fifth frame members extend from the third frame member to the second ring.

Preferably, the second, fourth and fifth frame members are linear.

Typically:
the first balloon is adhered to the first frame member; and
the second balloon is adhered to the second frame member.

Generally:
between each alternating turn of the first frame member, the first balloon is adhered to the first frame member at at least 3 spots that are spaced from each other; and
between each alternating turn of the third frame member, the second balloon is adhered to the third frame member at at least 3 spots that are spaced from each other.

Preferably:
the first balloon defines an inlet at a first axial end of the first balloon, in use, to inflate the first balloon; and
the second balloon defines an inlet at a first axial end of the second balloon, in use, to inflate the second balloon.

Typically:
the first balloon defines an inlet at a second axial end of the first balloon, in use, to inflate the first balloon; and
the second balloon defines an inlet at a second axial end of the second balloon, in use, to inflate the second balloon.

Generally, the first balloon and the second balloon are in fluid communication with each other, in use, to equalise pressure within the first and second balloons.

Preferably:
when the first balloon is in the expanded condition, the first balloon defines an angle of at least 30 degrees at each turn of the first balloon; and
when the second balloon is in the expanded condition, the second balloon defines an angle of at least 30 degrees at each turn of the second balloon.

Typically:
when the first balloon is in the expanded condition, at least 60% of the axial length of the first balloon is laterally spaced from adjacent portions of the first balloon; and
when the second balloon is in the expanded condition, at least 60% of the axial length of the second balloon is laterally spaced from adjacent portions of the second balloon.

Generally, both the first and second balloons are disposed radially within the frame.

Preferably, the first and second balloons are made of polyester (e.g. polyethelene terephthalate, PET), polyamide (e.g. PA 12) or polytetrafluoroethylene (PTFE).

Typically, the frame is made of nitinol.

### BRIEF DESCRIPTION OF THE DRAWINGS

The invention will now be described in more detail, by way of example only, with reference to the accompanying drawings in which:

FIG. 1 is a perspective view of an inflatable dilatation device according to a preferred embodiment of the invention, in a contracted condition;

FIG. 2 is a perspective view of the inflatable dilatation device in FIG. 1, in an expanded condition; and

FIG. 3 is a perspective view of an inflatable dilatation device according to an alternative embodiment of the invention, in an expanded condition.

### DESCRIPTION OF THE INVENTION

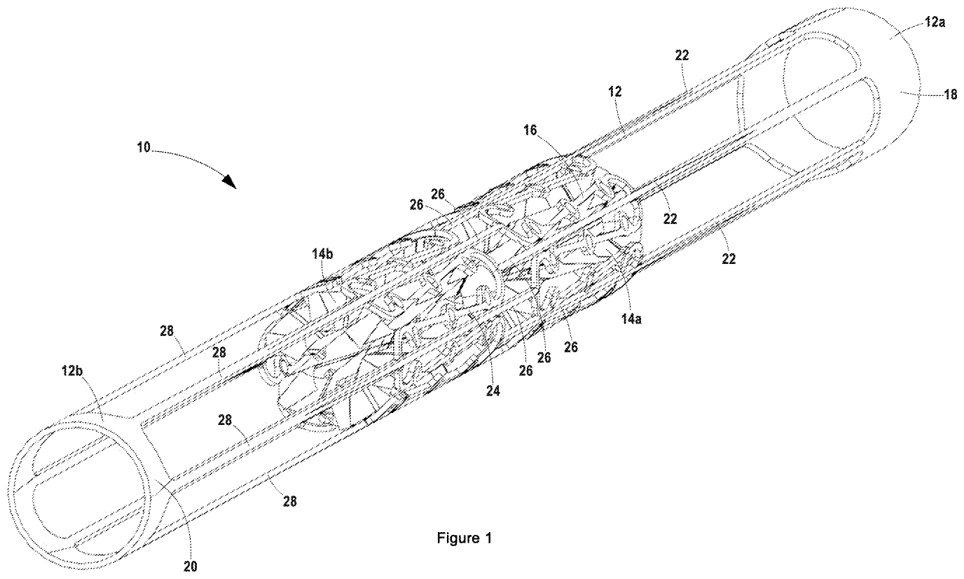

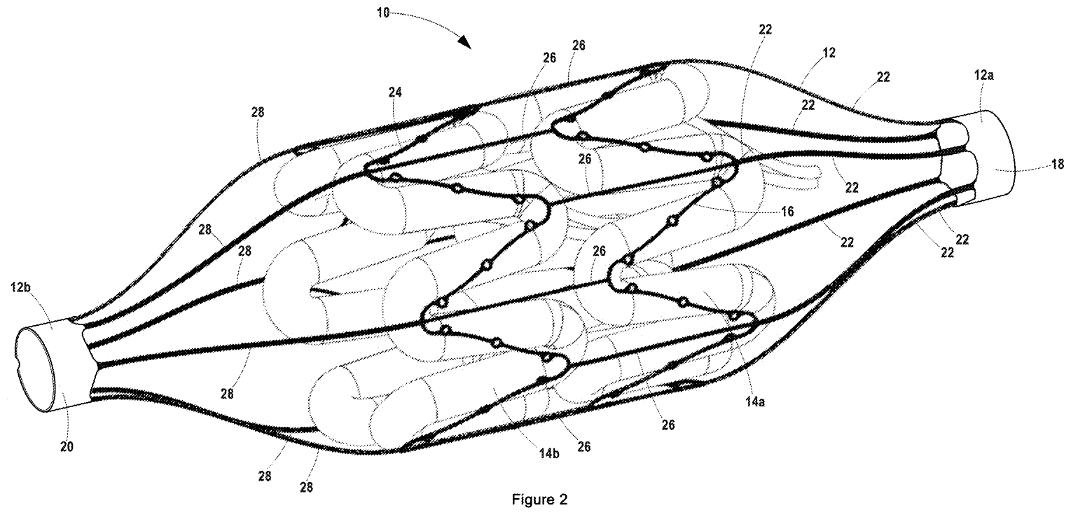

With reference to FIGS. 1 and 2 of the drawings, an inflatable dilatation device 10 includes a frame 12 and inflatable balloons 14.

In use, the dilatation device 10 may be used with a catheter (that includes pressurising means) to expand a stent within a patient's artery.

The frame 12 is tubular, defining first and second axial ends 12a and 12b, respectively. For convenience, the first axial end 12a will be referred to as the proximal end, and the second axial end 12b will be referred to as the distal end. The frame 12 includes:
A first member 16 that zigzags circumferentially. In this specification, the term “zigzag” is intended to mean “a line or course having alternate right and left turns”. The left and right turns need not be abrupt; a sinusoidal form is intended to be included in this definition. When the frame 12 is in a contracted condition, each “turn” is an acute-angle turn, defining an apex at each turn.
A first ring 18 at a first axial end 12a (i.e. proximal end) of the tubular frame 12.
A second ring 20 at a second axial end 12b (i.e. distal end) of the tubular frame 12.
A set of linear second members 22 that extend from apexes on a first side (i.e. a proximal side) of the first frame member 16 longitudinally in a first axial direction (i.e. towards the first axial end 12a (i.e. proximal end) of the frame 12) to the first ring 18. For clarification: “the first side of the first member 16” is intended to refer to the side of the first member 16 that is formed by apexes proximal the first axial end 12a of the frame 12; and “the second side of the first member 16” is intended to refer to the side of the first member 16 that is formed by apexes proximal the second axial end 12b of the frame 12. Furthermore, in this specification, the phrase “extends longitudinally” is intended to mean “extends towards an axial end of the frame 12”, and is not limited to second members 22 that extend axially, parallel to each other.
A third member 24 that: zigzags circumferentially; and is axially spaced from the second side (i.e. the distal side) of the first frame member 16. As shown in FIGS. 1 and 2: the apexes on a first side (i.e. proximal side) of the first frame member 16 are longitudinally/axially aligned with the apexes on a first side (i.e. proximal side) of the third frame member 24; and the apexes on a second side (i.e. distal side) of the first frame member 16 are longitudinally/axially aligned with the apexes on a second side (i.e. distal side) of the third frame member 20. As with the first frame member 16, the first side of the third member 24 is similarly intended to refer to the side of the third member 24 that is formed by apexes proximal the first axial end 12a of the frame 12; and the second side of the third member 24 is intended to refer to the side of the third member 24 that is formed by apexes proximal the second axial end 12b of the frame 12.
A set of linear fourth members 26 that extend longitudinally between axially aligned apexes defined by the first and third frame members 16 and 24. Put another way, the set of fourth members 26 extend between longitudinally/axially aligned apexes on a first side (i.e. a proximal side) of the first and third frame members 16 and 24; and longitudinally aligned apexes on a second side (i.e. a distal side) of the first and third frame members 16 and 24.
A set of linear fifth members 28 that extend from apexes on a second side (i.e. distal side) of the third frame member 24 longitudinally in a second axial direction (i.e. towards the second axial end 12b (i.e. distal end) of the frame 12) to the second ring 20.

Preferably, the first, second third, fourth and fifth members 16, 22, 24, 26 and 28 are laser cut from a single nitinol tube.

It will be appreciated that the rings 18 and 20 are not able to expand in sympathy with expansion of the first and/or third frame members 16 and 24, upon expansion of the frame 12 from a contracted condition shown in FIG. 1 to an expanded condition shown in FIG. 2.

Each balloon 14 is elongate—being initially formed as a linear, cylindrical balloon—and is made of polyester (e.g. polyethelene terephthalate, PET), polyamide (e.g. PA 12) or polytetrafluoroethylene (PTFE). Both balloons 14 are disposed radially within the frame 12. A first balloon 14a is secured to the first frame member 16, and a second balloon 14b is secured to the third frame member 24. More specifically: the first balloon 14a is adhered to the first frame member 16 at at least three spots (that are spaced from each other) between each adjacent pair of apexes defined by the first frame member 16 (i.e. between each alternating turn of the first frame member 16); and the second balloon 14b is adhered to the third frame member 24 at at least three spots (that are spaced from each other) between each adjacent pair of apexes defined by the third frame member 24 (i.e. between each alternating turn of the third frame member 24). The balloons 14a and 14b follow their respective frame members 16 and 24, thereby assuming a corresponding zigzag shape, with the balloons 14 defining elbows at the apexes of the first and third frame members 16 and 24.

It will be appreciated that: elbows (otherwise referred to as apexes) on both sides (i.e. the proximal and the distal sides) of the first balloon 14a correspond to the apexes on both sides (i.e. the proximal and distal sides) of the first frame member 16; and elbows (otherwise referred to as apexes) on both sides (i.e. the proximal and the distal sides) of the second balloon 14b correspond to the apexes on both sides (i.e. the proximal and distal sides) of the third frame member 24.

Upon inflation (i.e. pressurisation) of the balloons 14, increased pressure at the elbows defined by the balloons 14 induce the balloons to “straighten”, thereby circumferentially expanding the balloons 14 from a contracted condition shown in FIG. 1 to an expanded condition shown in FIG. 2. Since the balloons 14 are secured to the first and third frame members 16 and 24, expansion of the balloons 14 from the contracted condition to the expanded condition causes circumferential expansion of the first and second frame members 16 and 24 (and, consequently, radial expansion of the frame 12 and the dilation device 10) in unison with the first and second balloons 14a and 14b from the contracted condition shown in FIG. 1 to the expanded condition shown in FIG. 2.

In respect of each balloon 14, when the balloon 14 is in the expanded condition: the balloon defines an (internal) angle of at least 30 degrees (preferably, 40 degrees) at each turn of the balloon (i.e. at each elbow/apex); and at least 60% (preferably, 65%) of the axial length of the balloon 14 is laterally spaced from (i.e. not in contact with) adjacent portions of the balloon 14. When the balloon 14 is in the contracted condition, the balloon 14 defines an (internal) angle of less than 5 degrees (preferably, zero degrees) at each turn of the balloon (i.e. at each elbow/apex) (i.e. adjacent portions of the balloon 14 run parallel to each other).

Each balloon 14 defines an inlet at each axial end of the balloon 14, in use, to pressurise the balloon 14. Furthermore, both balloons 14a and 14b are in fluid communication with each other (via a duct), in use, to equalise pressure within the balloons 14a and 14b.

Since the first and second balloons 14a and 14b on the one hand and the first and third frame members 16 and 24 on the other hand zigzag circumferentially and follow each other, respectively, it will be appreciated that expansion of the dilatation device 10 from the contracted condition to the expanded condition does not induce significant relative movement of the first and second balloons 14a and 14b and their respective first and third frame members 16 and 24. This reduction in relative movement between the balloons 14 on the one hand and the first and third frame members 16 and 24 on the other hand enables the balloons 14 stably to be supported by the first and third frame members 16 and 24 alone. In other words, in respect of each balloon 14: no links are required to connect portions of the balloon 14 to other portions of the balloon 14; and portions of the balloon 14 between elbows/apexes need not contact adjacent portions of the balloon 14, to maintain stability.

According to a non-limiting embodiment of the inflatable dilatation device 10:
the frame 12 is:
   made of superelastic Nitinol;
   tubular with a diameter of 5.6 mm and with a wall thickness of 0.229 mm;
   laser cut to define the: first member 16, first and second rings 18 and 20, second members 22, third member 24, fourth members 26 and fifth members 28; and
   electropolished and smoothed;
the inflatable balloons 14:
   are made of a non-compliant Nylon 12 material;
   have an outer diameter of 5 mm;
   have a single wall thickness of 17 micron;
   has a total inflatable length of 252 mm and a zig-zag length of 21 mm;
   comprise two zig-zagging inflatable balloons 14 that are axially spaced from each other, each zig-zagging inflatable balloon 14 defining six elbows;
   are attached to the frame 12 by means of a cyanoacrolate glue; and
   each has necked-down sections with an outer diameter of 1.2 m preferably two such neck-down sections per inflatable balloon 14.

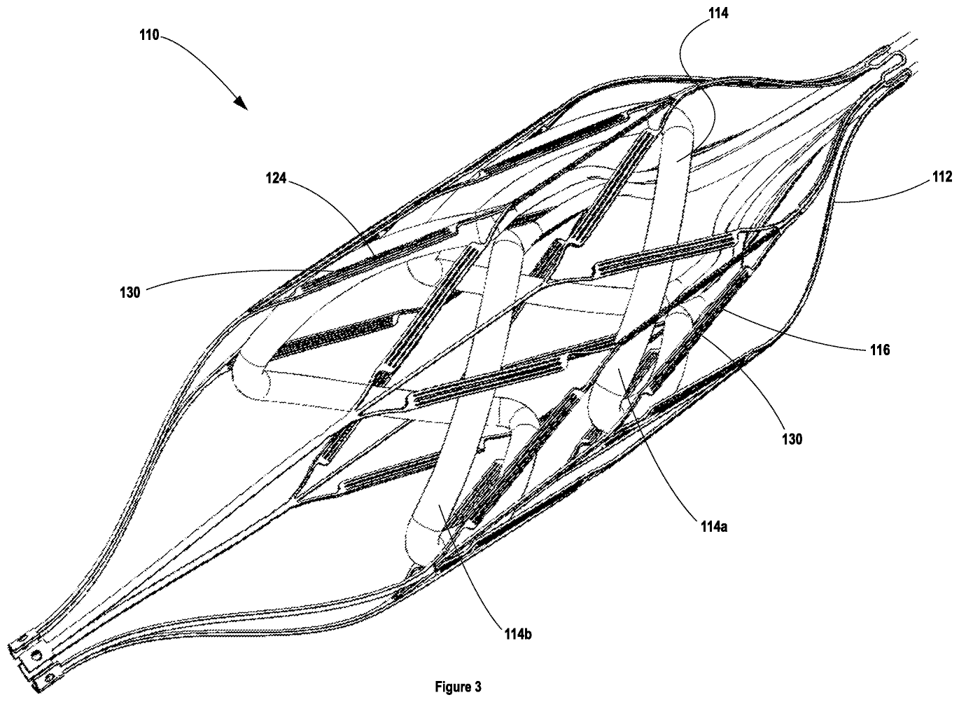

An inflatable dilatation device 110 according to an alternative (less preferred) embodiment of the invention is shown in FIG. 3, in an expanded condition. The dilatation device 110 according to the alternative embodiment of the invention is generally similar to the dilatation device 10 according to the preferred embodiment of the invention shown in FIGS. 1 and 2. However, according to this alternative embodiment 110 of the dilatation device (which similarly includes a frame 112 and inflatable balloons 114):
A first member 116 of the frame 112 zigzags circumferentially, which zigzags have a first wavelength.
A third member 124 of the frame 112 zigzags circumferentially, which zigzags have a wavelength similar to the first wavelength.
A first balloon 114a is secured to the first frame member 116, which first balloon 14a zigzags circumferentially, with the zigzags defined by the first balloon 114a having a second wavelength that is greater than the first wavelength of the first frame member 116 zigzags. More specifically, the second wavelength of the first balloon 114a is approximately three times the first wavelength of the first frame member 116 zigzags. It will be appreciated that, on each side of the first balloon 114a and the first frame member 116, the apexes of the first balloon 114a coincide with and are adhered to every third apex of first frame member 116.
Similarly, a second balloon 114b is secured to the third frame member 124, which second balloon 14b zigzags circumferentially, with the zigzags defined by the second balloon 114b having a second wavelength that is greater than the first wavelength of the third frame member 124 zigzags. More specifically, the second wavelength of the second balloon 114b is approximately three times the first wavelength of the third frame member 124 zigzags. It will be appreciated that, on each side of the second balloon 114b and the third frame member 124, the apexes of the second balloon 114b coincide with and are adhered to every third apex of third frame member 124.

Optionally, the first inflatable balloon 114a could be adhered to the first frame member 116 at further points of intersection of the first inflatable balloon 114a and the first frame member 116, between the apexes of the first inflatable balloon 114a/the first frame member 116.

Similarly, the second inflatable balloon 114b could optionally be adhered to the third frame member 124 at further points of intersection of the second inflatable balloon 114b and the third frame member 124, between the apexes of the second inflatable balloon 114b/the third frame member 124.

It will also be appreciated (albeit that this embodiment is not shown) that instead of the second wavelength of the first and second balloons 114a and 114b being three times the first wavelength of the first and third frame members 116 and 124, respectively, the first wavelength of the first and third frame members 116 and 124 could be three times the second wavelengths of the first and second balloons 114a and 114b, respectively. In such an embodiment, in respect of each side of the first and third frame members 116 and 124 and the first and second balloons 114a and 114b, apexes of the first and third frame members 116 and 124 could coincide with and be adhered to every third apex of the first and second balloons 114a and 114b, respectively.

Comparing the dilatation device 10 according to the preferred embodiment of the invention and the dilation device 110 according to the alternative embodiment of the invention, it will further be appreciated that:
the apexes defined by the first and third frame members 116 and 124 according to the alternative embodiment of the invention are more sharp/acute than the apexes defined by the first and third frame members 16 and 24 according to the preferred embodiment of the invention.
The first and third frame member 116 and 124 according to the alternative embodiment of the invention further define saddles 130 that increase the surface area of the first and third frame members 116 and 124 along portions of their length spaced from the apexes. These saddles 130 provide increased surface area for the first and second inflatable balloons 114a and 114b to adhere to the first and third frame members 116 and 124, respectively.

1. An inflatable dilatation device including:
a tubular frame including a first frame member that zigzags circumferentially; and
an elongate inflatable first balloon that:
is secured to the first frame member; and
zigzags along the first frame member,
such that inflation of the first balloon causes the first balloon and the first frame member circumferentially to expand in unison from a contracted condition to an expanded condition.

2. The inflatable dilatation device according to claim 1, wherein the frame further includes a set of second frame members that extend longitudinally in a first axial direction from apexes on a first side of the first frame member.

3. The inflatable dilatation device according to claim 2, wherein the frame further includes a third frame member that: zigzags circumferentially; and is axially spaced from the second side of the first frame member.

4. The inflatable dilatation device according to claim 3, wherein the frame further includes a set of fourth frame members that extend longitudinally between: longitudinally aligned apexes on a first side of the first and third frame members; and longitudinally aligned apexes on a second side of the first and third frame members.

5. The inflatable dilatation device according to claim 4, further including an elongate inflatable second balloon that:
is secured to the third frame member; and
zigzags along the third frame member,
such that inflation of the third balloon causes the third balloon and the third frame member circumferentially to expand in unison from a contracted condition to an expanded condition.

6. The inflatable dilatation device according to claim 5, wherein the frame further includes a set of fifth frame members that extend longitudinally in a second axial direction from apexes on a second side of the third frame member.

7. The inflatable dilatation device according to claim 6, wherein:
the apexes on both sides of the first balloon correspond to the apexes on both sides of the first frame member; and
the apexes on both sides of the second balloon correspond to the apexes on both sides of the third frame member.

8. The inflatable dilatation device according to claim 7, wherein the tubular frame further includes:
a first ring at a first axial end of the tubular frame; and
a second ring at a second axial end of the tubular frame,
which first and second rings do not expand in sympathy with expansion of the first or second frame members.

9. The inflatable dilatation device according to claim 8, wherein:
the set of second frame members extend from the first frame member to the first ring; and
the set of fifth frame members extend from the third frame member to the second ring.

10. The inflatable dilatation device according to claim 9, wherein the second, fourth and fifth frame members are linear.

11. The inflatable dilatation device according to claim 10, wherein:
the first balloon is adhered to the first frame member; and
the second balloon is adhered to the second frame member.

12. The inflatable dilatation device according to claim 11, wherein:
between each alternating turn of the first frame member, the first balloon is adhered to the first frame member at at least 3 spots that are spaced from each other; and
between each alternating turn of the third frame member, the second balloon is adhered to the third frame member at at least 3 spots that are spaced from each other.

13. The inflatable dilatation device according to claim 12, wherein:
the first balloon defines an inlet at a first axial end of the first balloon, in use, to inflate the first balloon; and
the second balloon defines an inlet at a first axial end of the second balloon, in use, to inflate the second balloon.

14. The inflatable dilatation device according to claim 13, wherein:
the first balloon defines an inlet at a second axial end of the first balloon, in use, to inflate the first balloon; and
the second balloon defines an inlet at a second axial end of the second balloon, in use, to inflate the second balloon.

15. The inflatable dilatation device according to claim 14, wherein the first balloon and the second balloon are in fluid communication with each other, in use, to equalise pressure within the first and second balloons.

16. The inflatable dilatation device according to claim 15, wherein:
when the first balloon is in the expanded condition, the first balloon defines an angle of at least 30 degrees at each turn of the first balloon; and
when the second balloon is in the expanded condition, the second balloon defines an angle of at least 30 degrees at each turn of the second balloon.

17. The inflatable dilatation device according to claim 16, wherein:
when the first balloon is in the expanded condition, at least 60% of the axial length of the first balloon is laterally spaced from adjacent portions of the first balloon; and
when the second balloon is in the expanded condition, at least 60% of the axial length of the second balloon is laterally spaced from adjacent portions of the second balloon.

18. The inflatable dilatation device according to claim 17, wherein both the first and second balloons are disposed radially within the frame.

19. The inflatable dilatation device according to claim 18, wherein the first and second balloons are made of polyester, polyamide or polytetrafluoroethylene.

20. The inflatable dilatation device according to claim 19, wherein the frame is made of nitinol.
